(19) United States
(12) Patent Application Publication
(10) Pub. No.: US 2022/0000537 A1
(43) Pub. Date: Jan. 6, 2022

(54) Devices and Methods for Electrosurgical Navigation

(71) Applicant: Baylis Medical Company Inc., Montreal (CA)

(72) Inventors: Gareth Davies, Toronto (CA); Ellen Harfield, Rockwood (CA); Rodney Horton, Austin, TX (US); David Wells, Etobicoke (CA); Jason Woo, Mississauga (CA)

(21) Appl. No.: 17/480,804

(22) Filed: Sep. 21, 2021

Related U.S. Application Data
(62) Division of application No. 15/532,402, filed on Jun. 1, 2017.
(60) Provisional application No. 62/087,141, filed on Dec. 3, 2014.

(51) Int. Cl. A61B 18/12; A61B 34/20; A61B 5/304

(52) U.S. Cl. CPC A61B 18/1233; A61B 18/1477; A61B 5/304; A61B 34/20

(57) ABSTRACT

A method and apparatus or system are disclosed for a procedure with minimal or no fluoroscopy, for example an electrosurgical procedure, and which uses a three dimensional mapping system. The procedure typically involves electrical measurement of the electrode of a needle to determine its position. Some embodiments further include intracardiac echocardiography (ICE) for tracking devices. The apparatus includes a needle with a tip electrode, an electroanatomical mapping (EAM) system, and an electrical generator, wherein a switching device is used to restrictively electrically connect the needle to only one of the mapping system or generator at a given time.

### RELATED APPLICATIONS

This application is a divisional of U.S. application Ser. No. 15/532,402, filed Jun. 1, 2017, which is a national phase application of international application PCT/IB2015/059337 which claims the benefit of the filing date of U.S. Provisional Application Ser. No. 62/087,141, filed Dec. 3, 2014, entitled “Devices and Methods for Electrosurgical Navigation”. All of the aforementioned application are hereby incorporated by reference in their entirety.

### TECHNICAL FIELD

The disclosure generally relates to methods and devices usable to deliver energy within the body of a patient. More specifically, the present disclosure is concerned with imaging and positioning of energy delivery devices within the body of a patient.

### BRIEF DESCRIPTION OF THE DRAWINGS

In order that the invention may be readily understood, embodiments of the invention are illustrated by way of examples in the accompanying drawings, in which:

FIG. 1 is a block diagram illustrating an embodiment of a system in accordance with the present invention;

FIG. 2 is an illustration of the steps of a method in accordance with the invention;

FIG. 3 is an illustration of an embodiment of a needle with a curved end portion and a handle;

FIG. 4 is an illustration of detail 4 of FIG. 3;

FIG. 5 is an illustration of detail 5 of FIG. 4;

FIG. 6 is an illustration of an embodiment wherein a switch box and an EAM system are connected using two pin blocks;

FIG. 7 is an illustration of an embodiment wherein a switch box and an EAM system are connected using one pin block; and

FIG. 8A to 8E are illustrations showing different views of an electrosurgical switch box.

### DETAILED DESCRIPTION

Typical transseptal procedures include a delivery of harmful X-rays for extended periods of time for fluoroscopy. This exposes both the patient and physician performing the procedure to the X-rays. The repeated exposure to x-rays of a physician that regularly performs such procedures is a significant concern. The physician can reduce exposure to the X-rays by wearing a lead apron, but this approach has the drawbacks of reduced maneuverability and possible back problems.

A transseptal procedure which minimizes or does not require fluoroscopy would avoid exposing the physician and patient to significant amounts of the associated X-rays. Disclosed herein are procedures which include using electroanatomical mapping (EAM) systems such as CARTO® (Biosense Webster) or NavX™ (St. Jude Medical), and other imaging tools, whereby the use of fluoroscopy is minimized or not required. Such systems may be used to provide localization of an electrosurgical device, such as, for example, a needle operable to deliver electrical energy through an electrode.

The present inventors have conceived of, and reduced to practice, embodiments of a system including a needle with an energy delivery electrode, an electroanatomical mapping (hereinafter, an “EAM”) system, and an electrical generator (i.e., a power supply), wherein a switch is used to restrictively electrically connect the needle to only one of the mapping system or generator at a given time. Such use of a switch prevents electrical communication between the mapping system and generator and thereby avoids associated problems. In some alternative embodiments, the system includes a relatively flexible energy delivery device (e.g., a wire with a distal electrode for delivering energy). In some embodiments, the power supply is a battery.

In one broad aspect, embodiments of the present invention comprise a transseptal procedure which uses an electroanatomical mapping system (i.e., a 3D (three dimensional) mapping system) and minimizes or substantially eliminates the need for fluoroscopy. In typical embodiments, the procedure includes using intracardiac echocardiography (ICE) for tracking devices. The procedure generally involves taking an electrical measurement of the electrode of an electrosurgical device, which includes measuring current, impedance and/or voltage. In some embodiments, a switch box is used to switch between delivering energy to a patient and sending sensory information to monitoring systems.

In a further broad aspect, embodiments of the present invention comprise a method of positioning an energy delivery device, for example a needle, using an electroanatomical mapping system to thereby minimize or eliminate the use of fluoroscopy.

In another broad aspect, embodiments of the present invention comprise a device for switching connectivity of a first device between two mutually incompatible devices (i.e., devices that cannot or should not be used at the same time) without having to disconnect any cables. In some of the embodiments, the first device is an energy delivery device configured for puncturing or ablating tissue and the other devices are a power source and an electrophysiological diagnostic system (hereinafter, an “EDS”). In some embodiments, the EDS is an electroanatomical mapping system. In some other embodiments, the EDS is an electrocardiogram (ECG) system.

In a further broad aspect, embodiments of the present invention comprise a dual mode cable apparatus comprising: a switch box; an electrosurgical cable for connecting an electrosurgical device, e.g. an electrosurgical needle, to the switch box; a generator cable for connecting a generator or other power supply to the switch box; and an EDS cable for connecting an EDS system (for example, an EAM system) to the switch box wherein the generator cable and EDS cable are usable sequentially but are generally not usable concurrently. The EDS cable may be referred to as an EAM cable hereinbelow but should be understood to be a cable that is operable to be connected to any electrophysiological diagnostic system or imaging system or the like. The generator cable is operable to deliver energy for puncturing or ablating tissue to the needle via the switch box. The EDS cable is operable to deliver sensory information from the needle to an EDS system via the switch box. The switch box is used to control whether the generator cable or the EDS cable are electrically connected to the needle at a given time.

In some alternative embodiments, the electrosurgical device is an ablation catheter, and the dual-mode cable apparatus comprises an ablation catheter cable in place of the electrosurgical cable. In some alternative embodiments, the EDS cable comprises a cable for connecting the switch box to an electrophysiological diagnostic system other than an electroanatomical mapping system wherein the cable is operable to deliver the appropriate signal to the electrophysiological diagnostic system (e.g., a signal for monitoring a position of a device, or sensing a parameter of a tissue). In some alternative embodiments, the power supply is a battery, and the dual-mode cable apparatus comprises a battery connection cable.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting. Also, it is to be further understood that while for illustrative purposes the drawings show a needle, other devices can be used with or comprise the system, apparatus, and methods.

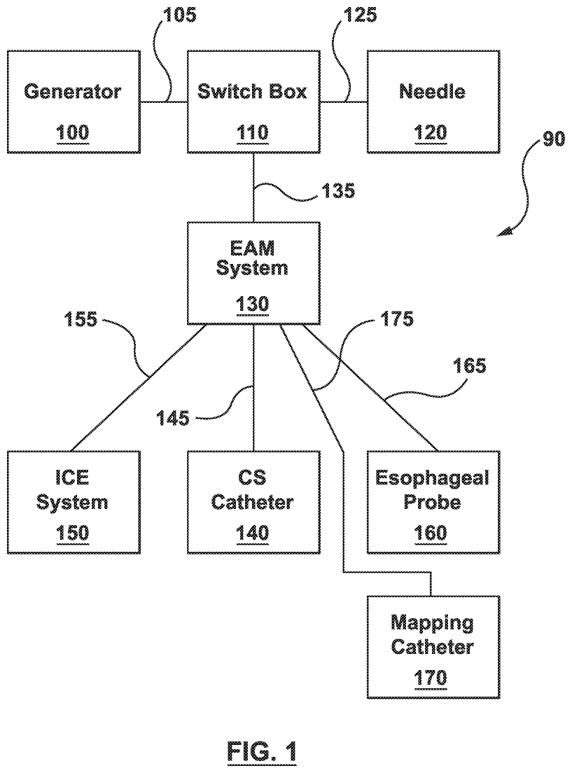

FIG. 1 is a block diagram illustrating an embodiment of a system 90. The system 90 includes an electroanatomical mapping system 130 (an EAM system) which is connected to: switch box 110 by EAM-switch box cable 135, CS catheter 140 (a coronary sinus catheter) by CS catheter cable 145, ICE system 150 by ICE system cable 155, esophageal probe 160 by esophageal probe cable 165, and to mapping catheter 170 by mapping catheter cable 175. Switch box 110 is further connected to generator 100 and electrosurgical needle 120 by generator cable 105 and needle cable 125, respectively.

Electroanatomical mapping system 130 is a 3D mapping system such as, for example, CARTO® (Biosense Webster) or NavX™ (St. Jude Medical). In some embodiments, the CARTO®3 system is used. The CARTO®3 system includes means for accepting impedance-based data for enabling catheters without magnetic coils to be visualized, and further includes means (the CARTOSOUND® Module) for enabling the visualization of anatomical landmarks mapped from an intracardiac echocardiography (ICE) system.

In typical embodiments, ICE system 150 is used to track devices up into the heart, and for monitoring the tenting and puncturing of a septum for gaining access to the left atrium.

In general, needle 120 is operable to conduct electricity and has an electrode at its distal end. In some embodiments, needle 120 comprises a metal shaft in which the shaft is covered with an electrical insulator and the tip of the shaft is electrically exposed (i.e., not covered by insulation) to form an active tip electrode. Some embodiments of needle 120 comprise an RF (radiofrequency) needle with a rounded, atraumatic tip which allows tracking the needle from the superior vena cava down into the right atrium while exposing the active tip electrode for visualization/localization using an electroanatomical mapping system 130 (e.g., CARTO®), without significant damage to the endocardium of the patient. An example of such a needle is the Baylis Medical NRG® transseptal needle. Further details regarding embodiments of a similar electrosurgical device are found in U.S. application Ser. No. 14/404,518, filed on Nov. 28, 2014, U.S. application Ser. No. 14/222,909, filed on Mar. 24, 2014, published as U.S. publication 2014/0206987, U.S. application Ser. No. 13/468,939, filed on May 10, 2012, which is now issued as U.S. Pat. No. 8,679,107, U.S. application Ser. No. 11/905,447, filed on Oct. 1, 2007, now U.S. Pat. No. 8,192,425, U.S. provisional application No. 60/827,452, filed on Sep. 29, 2006, and U.S. provisional application No. 60/884,285, filed on Jan. 10, 2007, all of which are herein incorporated by reference in their entirety.

Esophageal probe 160 of FIG. 1 typically contains an imaging element that may be visualized or localized using electroanatomical mapping system 130. In some embodiments, the imaging element is a decapolar catheter (or another catheter that may be visualized using the EAM system) which is contained in a lumen of the esophageal probe. An example of an esophageal probe is the 18 Fr Level 1 Acoustascope® Esophageal Stethoscope with Temperature Sensor, by Smiths Medical.

Esophageal probe 160 is used, for example, for monitoring the temperature inside the esophagus if the further procedural step of ablation within the left atrium is performed (once access has been gained to the left atrium). In typical embodiments of the method disclosed herein, gaining access to a left atrium by means of a transseptal puncture is achieved without using esophageal probe 160. Some alternative embodiments of system 90 that are used to gain transseptal access, but not for a further step of ablation within the left atrium, do not include esophageal probe 160.

Some embodiments of the EAM make use of a reference electrode for imaging/localization of needle 120. In some such embodiments, the reference electrode is one of the electrodes of a CS catheter 140 (a coronary sinus catheter), which may be a decapolar or duo-decapolar catheter. Typically, the proximal electrode of CS catheter 140 is used as the reference electrode. To function as a reference electrode for needle 120, the CS catheter is also connected to electroanatomical mapping system 130. As an example, embodiments of the method using a CARTO system may require a reference electrode. Other embodiments of the method, for example those using a NavX system, may not require a reference electrode. Consequently, some embodiments of system 90 do not include CS catheter 140.

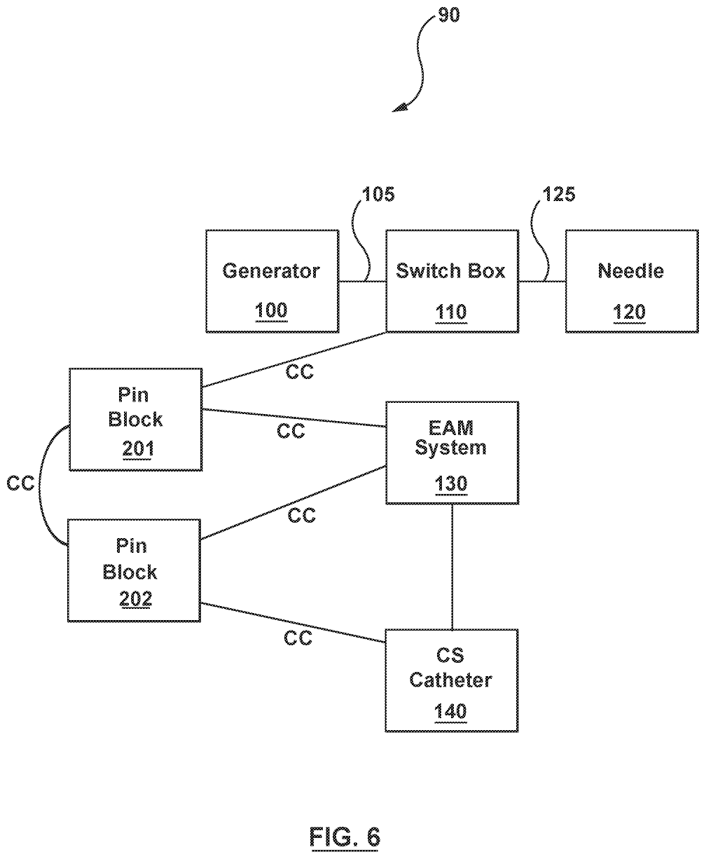

In one embodiment wherein EAM system 130 requires a reference electrode for imaging, switch box 110 and EAM system 130 are connected using two pin blocks. Making reference to FIG. 6, CS catheter 140 is connected to EAM system 130 through pin block 202. Typically, CS catheter 140 has 10 or 20 electrodes, each of which is connected to a pin connection of pin box 202. Switch box 110 is connected EAM system 130 through pin block 201. A jumper cable (or some other acceptable cable) connects one of the pin connectors of pin block 202 to pin block 201 to provide a reference electrode for needle 120. In FIG. 6, the connector cables connecting the components to the pin boxes (and the pin boxes to each other) are labeled as “CC”.

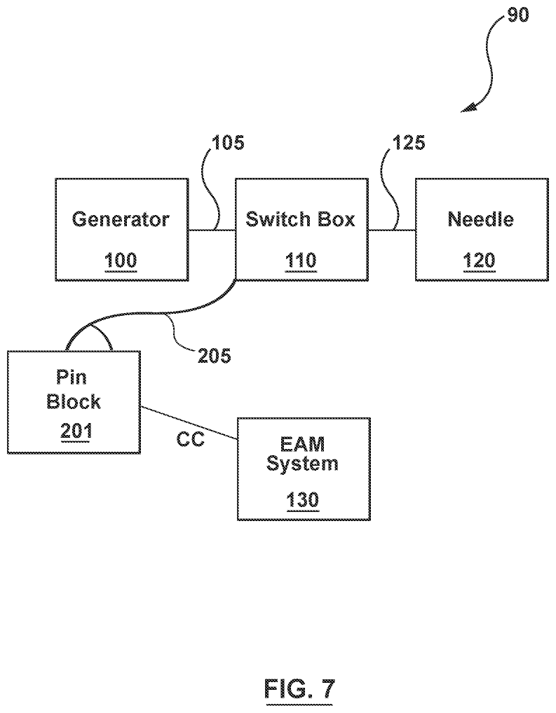

Another embodiment in which EAM system 130 requires a reference electrode for imaging, is illustrated in FIG. 7. The embodiment of FIG. 7 uses one pin box and does not require an additional catheter for providing a reference signal. In the example of FIG. 7, switch box 110 is connected to EAM system 130 through pin block 201 using splitter cable 205 whereby switch box 110 is connected to two pin connectors of pin box 201. The second connection provides a reference signal for imaging purposes. Some alternative embodiments do not use a splitter cable, but instead include switch box 110 being connected to one pin of pin block 201, and a jumper cable connecting the first pin to a second pin of pin block 201 to thereby provide a reference.

Making reference to FIG. 1, mapping catheter 170 may be, for example, an ablation catheter, a lasso catheter, or another type of catheter suitable for mapping.

As described above, switch box 110 is connected to: generator 100 by generator cable 105, needle 120 by needle cable 125, and electroanatomical mapping system 130 by EAM-switch box cable (or EDS cable) 135. As noted above, typical embodiments of needle 120 comprise an RF (radiofrequency) needle with a rounded, atraumatic tip, or another suitable electrosurgical device. Generator 100 provides energy to needle 120 (through switch box 110) enabling needle 120 to puncture tissue or to create a channel therethrough. Typical embodiments of generator 100 comprise an RF electrical generator.

The electroanatomical mapping system 130 enables the position of electrode 119 of needle 120 to be determined substantially in real time relative to the patient's anatomy and other procedural devices. In order to both be mapped and to be used for puncturing, needle 120 should be operable to be electrically connected to both the EAM system and to the RF generator. Switch box 110 functions to switch electrical connectivity of a device (e.g., needle 120) between two mutually incompatible devices (generator 100 and electroanatomical mapping system 130) without disconnecting any of the cables associated therewith, such that operation of the mapping system does not affect the RF generator, and operation of the RF generator does not affect the function of the mapping system. When connected through switch box 110, needle 120 is not operable to be electrically connected to both generator 100 and electroanatomical mapping system 130 simultaneously.

Typical embodiments of switch box 110 include a mechanical switch which is used by an operator to physically switch the needle between being electrically connected to the generator 100 or to the EAM system, depending upon procedural need. This mechanical switching prevents the RF generator and mapping system from negatively affecting the other's function, while allowing both to be used by the operator as needed. Switch box 110 thereby provides for a safe connection between needle 120, generator 100, and the electroanatomical mapping system 130.

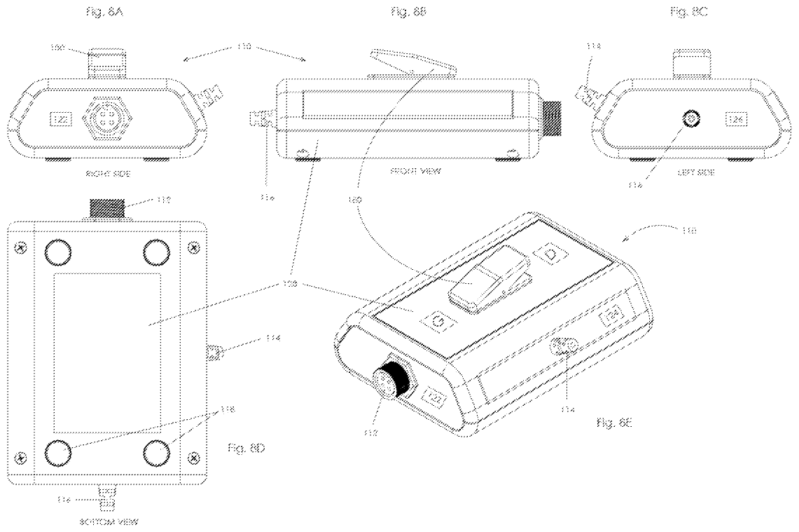

FIGS. 8A to 8E illustrate different views of an electrosurgical switch box. The electrosurgical switch box 110 comprises: a switch 180; a first connector 112 associated with the electrosurgical switch box for connecting an energy delivery device to the electrosurgical switch box 110; a second connector 114 associated with the electrosurgical switch box for connecting a power supply to the switch box; and a third connector 116 associated with the switch box for connecting an electrophysiological diagnostic system to the switch box. The switch 180 is operable in a first position, wherein the first connector 112 is electrically coupled to the second connector 114, and a second position, wherein the first connector is electrically coupled to the third connector 116. Typically, switch 180 is a rocker switch for providing visibility and the electrosurgical switch box includes a plurality of supports 118 configured to prevent slippage, which is beneficial in an operating room. In some embodiments, the first connector 112 is a four pin male connector and/or the second connector 114 is a four pin female connector and/or the third connector is a single pin connector. The electrosurgical switch box typically comprises a casing 128 wherein the casing is comprised of plastic which is an electrical insulator.

Typically, the electrosurgical switch box further comprises a plurality of markings for indicating a function of each of the connectors. Such markings may include a first marking 122 positioned proximate the first connector and illustrating an electrosurgical puncture device, a second marking 124 proximate the second connector and Illustrating a generator, and a third marking 126 proximate the third connector and illustrating a diagnostic system. Also, some embodiments include marking G illustrating a generator associated with the first position of the switch and a marking D illustrating a diagnostic system associated with the second position of the switch.

Some embodiments of system 90 include a dual-mode cable apparatus (or assembly) comprising: a switch box 110; a needle cable 125; a generator cable 105 and an EAM-switch box cable (or EDS cable, as noted above) 135. Switch box 110 is used to select which of generator cable 105 and EAM-switch box cable 135 is electrically connected to needle 120 at a given point in time.

In some embodiments, generator cable 105 comprises a conductive wire surrounded by a layer of insulation wherein the conductive wire and insulation are configured (e.g., to avoid dielectric breakdown) for delivering RF electrical energy to material such as tissue of a patient's body. Typically, a cable for supplying RF electrical energy (e.g. generator cable 105) to a patient for tissue puncturing or ablation has a thicker layer of insulation than a cable used for carrying diagnostic information (e.g. EAM-switch box cable 135) from a patient to a diagnostic device. Also, for safety reasons, a cable used for delivering electrical energy to a patient must undergo more rigorous testing than a cable used for communicating diagnostic information, and consequently, in general, cables used for delivering electrical energy to a patient for puncturing and ablation have a more rugged construction than cables used for diagnostic signals. Furthermore, in typical embodiments of system 90, a cable used for connecting to a power supply and a cable used for connecting to a diagnostic device have different plug configurations to ensure the correct use of cables, i.e., to avoid confusion and misconnection of devices. For example, in the embodiment of FIG. 1, generator cable 105 has a plug configured for connecting to generator 100 and EAM-switch box cable 135 has a plug configured for connecting to electroanatomical mapping system 130.

Typical embodiments of the dual-mode cable provide that a diagnostic signal can flow from an energy delivery device (e.g. an electrosurgical device such as a needle or an ablation catheter) to a diagnostic device or system, and that an electrical flow of sufficient power for treating a patient can flow from a power supply to the energy delivery device.

Some alternate embodiments include a switching system (switch box 110) which not only switches the RF device (e.g., needle 120) electrical connection between the mapping system and the generator, but also initiates RF energy delivery by the generator and the RF device when the switch electrically couples needle 120 to generator 100. In some such embodiments, the connection of generator 100 to needle 120 includes an initiation of RF energy delivery, and then, when energy delivery is complete, the system automatically connects needle 120 to the mapping system.

Another alternate embodiment of switch box 110 includes an electronic circuit (passive or active) for electrically decoupling and isolating electroanatomical mapping system 130 from generator 100, while allowing needle 120 to be electrically and functionally connected to both.

In general, embodiments of the present invention comprise a device (e.g., switch box 110) for switching connectivity of a first device between two mutually incompatible devices (i.e., devices that should not be used at the same time) without having to disconnect any cables. In some of the embodiments, the first device is an energy delivery device configured for puncturing or ablating tissue (e.g. needle 120), and the other devices are a power source (e.g. generator 100) and an electrophysiological diagnostic system (an EDS). In some embodiments, the EDS is an electroanatomical mapping system (e.g., Electroanatomical mapping system 130). In some other embodiments, the EDS is an ECG system, or another electrophysiological diagnostic system known to those skilled in the art. In some embodiments, the electrophysiological diagnostic system is used for sensing, locating or characterizing abnormal cardiac tissue (e.g., localizing the focal source of an atrial fibrillation). In alternate embodiments of system 90, the first device may be usable at locations within a body of a patient other than in or near the heart.

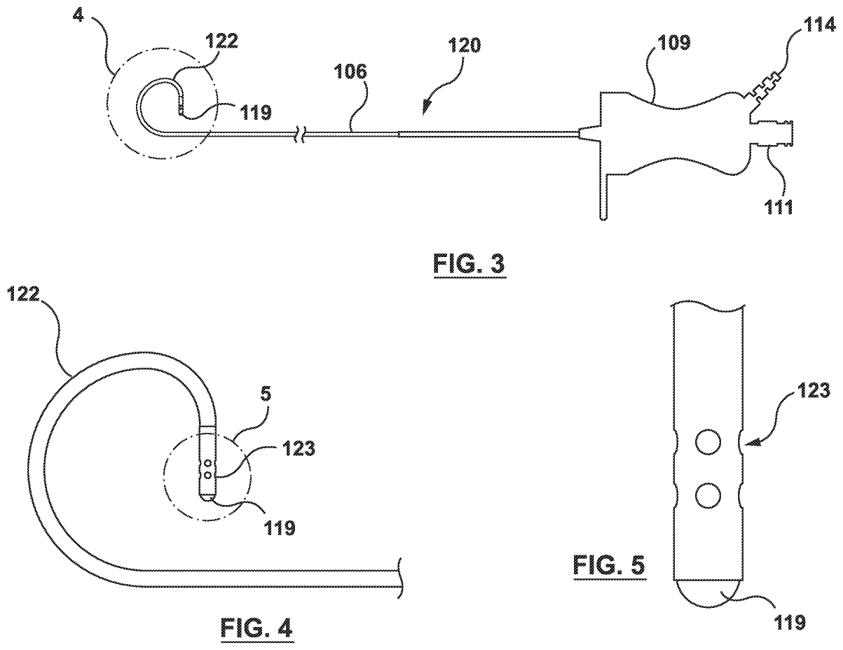

FIG. 3 is an illustration of an embodiment of an electrosurgical device such as needle 120 with a curved distal portion 122 and a handle 109. While the embodiment of FIG. 3 has distal curve of 270 degrees, in some alternative embodiments the distal portion is straight, and in other embodiments the distal portion has a curve between 0 and 270 degrees. Handle 109 includes electrical connector 114 and fluid connector 111. Needle 120 includes shaft 106 and electrode 119 at the end of curved distal portion 122. In typical embodiments shaft 106 includes a lumen (not shown in drawing) which is in fluid communication with fluid connector 111. FIG. 4 is an illustration of detail 4 of FIG. 3 which includes curved distal portion 122, electrode 119 and apertures 123. Electrode 119 is in electrical communication with electrical connector 114, and may be used for delivering electrical energy to the tissue of a patient. FIG. 5 is an illustration of detail 5 of FIG. 4 which includes electrode 119 and apertures 123. Apertures 123 are in fluid communication with the lumen of shaft 106, and may be used for delivering fluids, withdrawing fluids, or measuring pressure.

In another aspect of the present invention, embodiments of a method for gaining access to a left atrium are disclosed. Generally, such embodiments comprise the steps of: (a) using intracardiac echocardiography (ICE) and an ICE catheter for visualization (or an ablation catheter or other catheter that can be visualized on the electroanatomical mapping system) while advancing a guidewire and a sheath up the right femoral vein and inferior vena cava, and into the right atrium; (b) using ICE and a mapping catheter for mapping the right side (or just the right atrium) of the heart; (c) advancing an electrosurgical puncturing device (e.g. an RF needle) and a dilator through the inferior vena cava (IVC) and into the heart; (d) positioning the tip of the needle outside of the dilator whereby its distal tip electrode is exposed, and using an electroanatomical mapping system for electrical based monitoring (i.e. measuring current, impedance, and/or voltage) to visualize/determine the position of the tip of the needle; (e) positioning the tip of the needle at the target tissue (e.g. the fossa ovalis); (f) using ICE to confirm tenting of the target tissue; (g) using a switch box to disconnect or decouple the needle from the electroanatomical mapping system and connecting or coupling it to a generator; and (h) delivering energy from the generator through the electrode at the tip of the needle to puncture the target tissue. The method typically further includes, as necessary, switching back to couple the needle to the electroanatomical mapping system. In some embodiments of a method aspect of the present invention, one or more of these steps may be absent or altered.

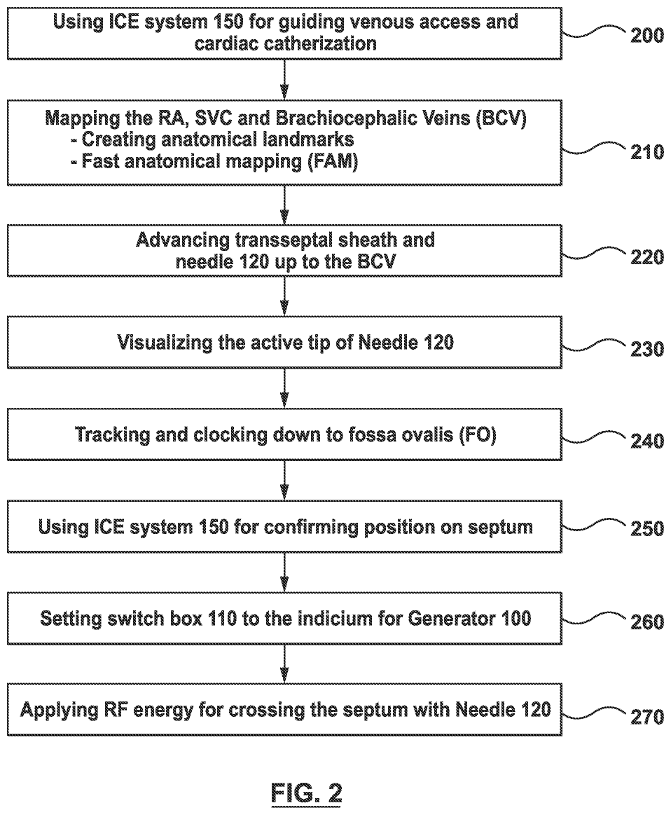

FIG. 2 is an illustration of the steps of a more specific method for gaining access to a left atrium. Typically, the switch box 110 is set to the EDS/EAM (mapping) mode for sensing or mapping at the start of the procedure. The first step of the method, step 200, comprises using ICE system 150 for guiding venous access and cardiac catheterization. Step 200 includes gaining access to the right and left femoral veins, introducing an ICE catheter into the left femoral vein access location, and introducing guidewires and sheaths into the right femoral vein access location. Step 200 further includes using ICE for confirming correct tracking of the guidewires and sheaths up the inferior vena cava (IVC), for example at the following checkpoints: (1) the bifurcation of the iliac veins (the inferior most portion of the IVC); (2) half-way up the IVC; and (3) within the right atrium (RA).

An alternative way of performing step 200 comprises advancing a transseptal sheath with an ablation catheter (or other catheter that can be visualized on the electroanatomical mapping system) by advancing the sheath, while the catheter is extended just beyond the tip of the sheath to allow visualization of the tip of the catheter, to thereby guide the transseptal sheath from the femoral vein, up the inferior vena cava, and into the right atrium.

Step 210 comprises mapping the RA, the superior vena cava (SVC), the brachiocephalic veins (BCV), and the superior most portion of the inferior vena cava. Step 210 includes creating anatomical landmarks by using ICE for identifying key anatomical features and displaying the anatomical features on the EAM screen (e.g. a CARTO® screen image). In some embodiments of the method, the key anatomical features include: (a) the aortic root, (b) the fossa ovalis, and (c) the coronary sinus (CS). An additional part of step 210 comprises fast anatomical mapping (FAM) which includes advancing a transseptal sheath over the previously advanced guidewire up to the right atrium, withdrawing the guidewire and dilator, advancing a mapping catheter 170 which may be, for example, an ablation catheter or a lasso catheter, and using the mapping catheter 170 to map the right side of the heart. This typically includes mapping the right atrium, the superior vena cava, the right brachiocephalic vein, and the superior most portion of the inferior vena cava. In some alternative embodiments, only the right atrium is mapped.

Step 220 comprises advancing a transseptal sheath and inserting needle 120. Step 220 includes advancing the transseptal sheath up the superior vena cava to the brachiocephalic veins (BCV), advancing the mapping catheter 170 through the transseptal sheath and confirming position, removing the mapping catheter 170, and inserting and advancing the transseptal dilator and needle 120 through the transseptal sheath. Step 230 is for visualizing or locating the active tip of needle 120. Step 230 typically includes positioning electrode 119 at the tip of needle 120 to be electrically exposed (i.e., extending the tip of needle 120 out the distal end the dilator), and electrically monitoring electrode 119 using the EAM system.

Step 240 comprises tracking and clocking down to the fossa ovalis. Step 240 includes tracking the position of electrode 119 using the EAM for visualization, moving the needle tip down from the SVC, and positioning electrode 119 at the fossa ovalis. Step 250 comprises advancing electrode 119 against the fossa ovalis to cause tenting of the tissue into the left atrium. Step 250 further comprises using the ICE system 150 for confirming the position of electrode 119 on the septum, which includes using ICE for confirming the septum is tented in the correct orientation for applying RF energy for puncturing.

Step 260 includes setting switch box 110 to the setting or mode for coupling needle 120 to generator 100 which establishes electrical communication between generator 100 and needle 120. When needle 120 is connected for receiving energy from the generator, electrode 119 is no longer visible on the EAM screen/monitor or image. Step 270 includes applying RF energy for crossing the septum with needle 120, which comprises using generator 100 to supply energy to electrode 119 of needle 120 through switch box 110, puncturing the septum, and advancing needle 120 across the septum. Once the septum is punctured, switch box 110 is typically set to the mode for sensing or mapping and the location of the needle tip is confirmed using electroanatomical mapping system 130.

Embodiments of the method typically include the further steps of advancing a sheath into the left atrium and confirming the position of the sheath using ICE. Some embodiments of the method include the further steps of creating a second puncture, advancing a second sheath through the second puncture. Such embodiments may include marking the second sheath's position on ICE, and using the second puncture location (where the second sheath crosses the septum) as a guide for additional transseptal puncturing.

Certain embodiments of the method include advancing a mapping catheter 170 into the left atrium for mapping following the transseptal puncture. Some embodiments further include advancing an ablation catheter into the left atrium for ablation. Some additional embodiments further include further procedural steps such as advancing a pacemaker lead, mitral valve device, left atrial appendage device, heart monitor or another device into the left atrium.

Embodiments of the method that include the further step (after step 270) of ablating tissue in the left atrium typically include using esophageal probe 160 (and the catheter contained therein for visualization) for monitoring temperature in the esophagus during ablation to prevent undesired tissue damage. Prior to being used, esophageal probe 160 is inserted down the esophagus approximately to the level of the heart, typically before step 200 of the method shown in FIG. 2 i.e. before gaining access to the right and left femoral veins. Furthermore, once the LA is mapped out as described above, the position of the esophageal probe is adjusted, as needed, to ensure that it is in position around/at/about the ostia of the left pulmonary veins and left atrium.

Some embodiments of the method include a further step of using fluoroscopy to confirm the information gained by the other imaging methods used in the method. In some such embodiments, fluoroscopy is used more than once for confirming information. Typically, fluoroscopy is used for minimal periods of time (or, at least, an amount of time less than what would be used in a standard procedure lacking electroanatomical mapping).

In other embodiments, methods of the present invention may be used for treatment procedures involving other regions within the body, and the invention is not limited in this regard. For example, some embodiments of the devices, systems and methods of the present invention use a flexible elongate device for delivering energy, (e.g. the Nykanen Radiofrequency Wire (Baylis Medical Company Inc., Montreal, Canada)), whereby such embodiments may be used to treat pulmonary atresia.

In other applications, embodiments of a device of the present invention may be used to create voids or channels within or through other tissues of the body, for example within or through the myocardium of the heart. In other embodiments, the device may be used to create a channel through a fully or partially occluded lumen within the body. Examples of such lumens may include, but are not limited to, blood vessels, the bile duct, airways of the respiratory tract and vessels and/or tubes of the digestive system, the urinary tract and/or the reproductive system. In such embodiments, the device may be positioned such that an electrode of the device is substantially adjacent the material to be perforated. Energy may be delivered from an energy source, through the electrode 119, to the target site such that a void, perforation, or channel is created in or through the tissue.

As described hereinabove, embodiments of the present invention comprise a procedure which uses an electroanatomical mapping system or other suitable system or apparatus to minimize or eliminate the need for fluoroscopy during electrosurgical procedures. The procedure typically involves electrical measurement of a portion (e.g., an electrode) of a needle (e.g., measuring current, impedance and/or voltage) or other electrosurgical device. Some embodiments further include intracardiac echocardiography (ICE) for tracking devices. Some embodiments comprise a method of positioning an atraumatic needle using an electroanatomical mapping system with minimal or no fluoroscopy. Typical embodiments comprise a device for switching connectivity of an electrosurgical device between mutually in-compatible devices (i.e. devices that should not be used at the same time) without the need to disconnect any cables.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A method for gaining access to a region of a heart, the method comprising the steps of:
(a) advancing an electrosurgical puncturing device into the heart;
(b) visualizing a position of a tip of the electrosurgical puncturing device using an electroanatomical mapping system;
(c) positioning the tip of the electrosurgical puncturing device at a target tissue;
(d) actuating a switch to electrically decouple the electrosurgical puncturing device from the electroanatomical mapping system while electrically coupling the electrosurgical puncturing device to an electrical generator; and
(e) delivering energy from the electrical generator through an electrode at a tip of the electrosurgical puncturing device to puncture the target tissue.

2. The method of claim 1, wherein step (c) further comprises tenting the target tissue with the electrosurgical puncturing device and using intracardiac echocardiography to confirm tenting of the target tissue.

3. The method of claim 1, wherein step (c) further comprises advancing a tip of the electrosurgical puncturing device outside of a tubular support member, the tip comprising an electrode.

4. The method of claim 1, further comprising using the electroanatomical mapping system for electrically-based monitoring wherein the electrically-based monitoring includes measuring current, impedance, and/or voltage.

5. The method of claim 1, further comprising, prior to step (a), advancing a guidewire and a sheath through a right femoral vein and inferior vena cava, and into a right atrium while using intracardiac echocardiography for visualization.

6. The method of claim 1, further comprising, prior to step (a), mapping a right atrium of the heart using intracardiac echocardiography.

7. The method of claim 6, further comprising using a mapping catheter.

8. The method of claim 1, further comprising, prior to step (a), mapping a right side of the heart using intracardiac echocardiography.

9. The method of claim 8, further comprising using a mapping catheter for the step of mapping a right side of the heart.

10. The method of claim 1, further comprising identifying one or more key anatomical features using intracardiac echocardiography and displaying the key anatomical features on a display of the electroanatomical mapping system.

11. The method of claim 10, wherein the key anatomical features include: (a) an aortic root, (b) a fossa ovalis, and (c) a coronary sinus.

12. The method of claim 1, wherein step (c) includes:
advancing a transseptal sheath through a superior vena cava to a brachiocephalic vein;
advancing the electrosurgical puncturing device through the transseptal sheath; and
tracking and clocking down the electrosurgical puncturing device to the target tissue.

13. The method of claim 1, wherein the target tissue is a septum, and the method further comprises the steps of setting the switch to a mode for sensing or mapping, and confirming a location of a tip of the electrosurgical puncturing device using the electroanatomical mapping system.

14. The method of claim 1, wherein the target tissue is a septum, and wherein the method further comprises the steps of advancing a sheath into a left atrium through the puncture and confirming the position of the sheath using intracardiac echocardiography following the advancement.

15. The method of claim 1, wherein the target tissue is a septum, and wherein the method further comprises the steps of creating a second puncture and advancing a second sheath through the second puncture.

16. The method of claim 1, wherein the target tissue is a septum, and wherein the method further comprises a step of advancing a mapping catheter into a left atrium through the puncture.

17. The method of claim 1, wherein the target tissue is a septum, and wherein the method further comprises a step of advancing an ablation catheter into a left atrium through the puncture.

18. The method of claim 17, wherein the method further comprises the steps of inserting an esophageal probe through an esophagus and monitoring a temperature in the esophagus during the step of ablating to prevent undesired tissue damage.

19. The method of claim 1, wherein the electrosurgical puncturing device is an RF needle.

20. The method of claim 1, wherein the electrosurgical puncturing device comprises a wire operable to delivery RF energy.
